(19) United States
(12) Patent Application Publication
(10) Pub. No.: US 2012/0308488 A1
(43) Pub. Date: Dec. 6, 2012

(54) ORAL CARE PRODUCT FOR SENSITIVE ENAMEL CARE

(75) Inventors: Shira Pilch, Highland Park, NJ (US); James Gerard Masters, Ringoes, NJ (US); Zhi Lu, Philipsburg, NJ (US); Davide Miksa, Doylestown, PA (US); Venda Porter, Piscataway, NJ (US)

(73) Assignee: COLGATE-PALMOLIVE COMPANY, New York, NY (US)

(21) Appl. No.: 13/522,517

(22) PCT Filed: Jan. 28, 2011
(86) PCT No.: PCT/US11/22875

§ 371 Date: Jul. 17, 2012

Related U.S. Application Data
(60) Provisional application No. 61/299,637, filed on Jan. 29, 2010.

(51) Int. Cl. A61K 8/81; A61Q 11/00; A61C 17/00; A61K 8/27

(52) U.S. Cl. 424/49; 433/216

(57) ABSTRACT

Disclosed are anti-erosion oral care formulations and methods that provide erosion protection while maintaining adequate cleaning performance. The anti-erosion oral care formulations include a mucoadhesive polymer and a zinc compound or salt that becomes more soluble at acidic pH.

### CROSS REFERENCE TO RELATED APPLICATIONS

This application claims priority to U.S. Provisional Patent Application No. 61/299,637, filed on Jan. 29, 2010, which is incorporated herein by reference.

### FIELD OF THE INVENTION

These embodiments relate to anti-erosion oral care compositions that provide erosion protection while maintaining adequate cleaning performance.

### BACKGROUND OF THE INVENTION

The erosion of dental enamel can lead to pain, discoloration, mechanical failure, and greater susceptibility to dental carries. Chemical erosion of tooth enamel may arise from the presence of acid in the oral cavity. Saliva constituents, mainly proteins and minerals, along with the pellicle are integral in protecting against an erosive challenge. The minerals and proteins in saliva help provide a chemical barrier to slow down or shift the complex dynamic equilibrium of hard tissue demineralization, while the pellicle will provide a diffusion barrier to accomplish the same process.

An oral care composition may protect teeth in a variety of ways. Many oral care compositions are designed to increase the pH in the oral cavity. A common strategy when attempting to control oral pH is to include an alkaline agent in the formulation of the oral care composition. The alkaline agent reacts with acid to neutralize the acid, forming water and a salt. This process raises the pH in the oral cavity. However, even when the pH in the oral cavity is high, the pH at the surface of the teeth, where cariogenic bacteria may be present, may be locally lower than the oral cavity in general due to bacterial activity. Soluble bases are not able to preferentially locate at the tooth surface, where acid does the most damage to teeth.

A method for reducing the impact of acid based erosion on teeth is to add soluble metal ions into oral care compositions. Zinc and calcium ions have been used to bind to the surface of the tooth, increasing the resistance of the tooth to acid damage. Zinc salts have also been used as antibacterial, antitartar, and anticalculus agents. Soluble metal salts are not able to preferentially locate at the tooth surface, where acid does the most damage to teeth. Additionally, soluble zinc salts may lead to unpleasant taste characteristics in the oral care composition.

Insoluble zinc compounds have been investigated as metal sources that have a longer residence time in the oral cavity. Amine polymers have been utilized to increase the residence time of the zinc compounds in the oral cavity. These compositions increase the amount of time zinc ions are found in the saliva. The zinc ion concentration in saliva does not address the local zinc concentration at the surface of the tooth, where acid erosion takes place. Accordingly, there is a need for an improved dentifrice that can provide a zinc source over time located at the surface of a tooth to prevent acid based erosion.

### SUMMARY OF THE INVENTION

In accordance with a feature of an embodiment, there is provided a composition and method for the prevention and/or treatment of dental erosion comprising an oral care composition that includes an orally acceptable vehicle, a mucoadhesive polymer, a zinc compound or salt that becomes more soluble at acidic pH, and an abrasive. While not intending on being bound by any theory of operation, it is believed that application of the composition to the teeth is believed to protect tooth enamel from erosion by forming a barrier of polymer and zinc compound at the surface of a tooth. The zinc compound is eroded by acids, which substitutes for erosion that otherwise could occur at the tooth. In addition to helping spatially locate the zinc compound at the tooth surface, the inventors believe that the mucoadhesive polymer may also reduce bacterial adhesion at the surface of the tooth.

In accordance with an additional embodiment, the invention includes a method of reducing acid based erosion of teeth comprising administering an oral care composition comprised of an orally acceptable vehicle, a mucoadhesive polymer, a zinc compound or salt that becomes more soluble at acidic pH, and an abrasive, and optionally applying a shear stress to the composition to shear-align a layer of mucoadhesive polymer and zinc compound to make the layer more homogeneous. When the composition is applied using a shear stress, enhanced erosion protection is derived from the additional homogeneity of the composition.

### DETAILED DESCRIPTION

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The headings (such as “Background” and “Summary,”) used herein are intended only for general organization of topics within the disclosure of the invention, and are not intended to limit the disclosure of the invention or any aspect thereof. In particular, subject matter disclosed in the “Background” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. All references cited in the Description section of this specification are hereby incorporated by reference in their entirety.

The description and specific examples, while indicating embodiments of the invention, are intended for purposes of illustration only and are not intended to limit the scope of the invention. Recitation of multiple embodiments having stated features is not intended to exclude other embodiments having additional features, or other embodiments incorporating different combinations of the stated features. Specific Examples are provided for illustrative purposes of how to make, use and practice the compositions and methods of this invention and, unless explicitly stated to recite activities that have been done (i.e., using the past tense), are not intended to be a representation that given embodiments of this invention have, or have not, been performed.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. As used herein, the word “include,” and its variants, is intended to be non-limiting, such that recitation of items in a list is not to the exclusion of other like items that may also be useful in the materials, compositions, devices, and methods of this invention. In a similar manner, the description of certain advantages or disadvantages of known materials and methods is not intended to limit the scope of the embodiments to their exclusion. Indeed, certain embodiments may include one or more known materials or methods, without suffering from the disadvantages discussed herein.

As used herein, the term “comprising” means that other steps and other components that do not affect the end result may be utilized. The term “comprising” encompasses the expressions “consisting of,” and “consisting essentially of.” The expression “effective amount,” as used herein denotes an amount of a compound or composition sufficient to significantly induce a positive benefit, preferably an oral health benefit, but low enough to avoid serious side effects, i.e., to provide a reasonable benefit to risk ratio, within the sound judgment of a person having ordinary skill in the art. The use of singular identifiers such as “the,” “a,” or “an” is not intended to be limiting solely to the use of a single component, but may include multiple components.

All percentages and ratios used herein are by weight of the oral care composition, unless otherwise specified. All measurements are made at 25° C., unless otherwise specified.

Throughout this description and claims, the disclosure of a certain numerical value (e.g., temperature, weight percent of components, etc.) is meant to denote that value, plus or minus an additional value that would be understood by persons having ordinary skill in the art, depending on the variable and the degree of measurement error typically associated with that value. For example, a given temperature would be understood by a person having ordinary skill in the art to include up to 10% variability, given the instrument used to measure the temperature.

The dentifrice of the present invention combines an insoluble compound of zinc with a mucoadhesive polymer and an abrasive, all in an orally acceptable vehicle, to form compositions that combat acid based erosion of teeth. The mucoadhesive polymer localizes the insoluble zinc compound near the surface of the tooth. Mucoadhesives that have a longer residence time at the surface of the tooth offer enhanced protection. The mucoadhesive both localizes the metal compound and prevents the adhesion of bacteria on the tooth surface. The zinc compounds are believed to be eroded as the pH in the oral cavity drops, thus sacrificing the metal compound for the benefit of the tooth enamel. Acid erosion of the zinc compounds results in a neutralization of acid, in turn raising the pH near the surface of the tooth. The zinc ions at the tooth's surface also may function as an antibacterial for any carriogenic bacteria. By increasing the residence time of the insoluble zinc compound at the tooth surface, the reservoir of zinc ions may remain in place and active longer.

While not intending on being bound by any theory of operation, the inventors believe that the mucoadhesive polymer, when used in the compositions and methods described herein, prevents the adherence of carriogenic bacteria to the enamel, and has a long residence time at the enamel surface. It also is believed that the mucoadhesive polymer spatially confines some of the zinc compound to the surface of the tooth. As the local environment around the tooth becomes acidic, the zinc compound is believed to become more soluble. The inventors believe that the process of solubilizing the zinc compound both neutralizes acid and provides beneficial soluble zinc ions. The long residence time of the mucoadhesive at the enamel surface is believed to provide a longer residence time for the metal compounds.

The compositions and methods of use of the present invention also provide enhanced acid protection by forming more ordered surface films when subject to shear stress. Shear stress originating from acts such as brushing, scrubbing, rubbing with a finger, and the like, results in shear alignment of the composition. The expression “shear alignment” as used in the specification and claims refers to a process in which a material becomes, at least partially, more ordered in response to an applied shear stress. Shear alignment will be understood to create a more homogeneous composition as a result of at least partial ordering. In the present invention, improved ordering may occur due to the formation of a more regular array of polymer, a more regular array of metal in the polymer, or a combination of both more ordered polymer and metal.

To prepare an anti-erosive oral care composition of the present invention a mucoadhesive polymer, a zinc compound or salt that becomes more soluble at acidic pH, and an abrasive are incorporated into an orally acceptable vehicle.

The oral care compositions of the various embodiments preferably are in the form of a dentifrice. The term “dentifrice” as used throughout this description, denotes a paste, gel, or liquid formulation. The dentifrice may be in any desired form, such as toothpaste; (including deep striped, surface striped, multi-layered, having a gel surround the paste); powder; beads; mouthwash; mouth rinses; lozenge; dental gel; a periodontal gel; a liquid suitable for painting a dental surface; a chewing gum; a dissolvable, partially dissolvable or non-dissolvable film or strip; a wafer; a wipe or towelette; an implant; a foam; a troche; a dental floss or any combinations thereof. Preferably, the dentifrice is a toothpaste.

The expression “orally acceptable vehicle” used in the context of the present invention means any vehicle useful in formulating any of the dentifrices described above. Suitable orally acceptable vehicles include, for example, one or more of the following: a solvent, an alkaline agent, a humectant, a thickener, a surfactant, an abrasive, an anti-calculus agent, a colorant, a flavoring agent, a dye, a potassium containing salt, an anti-bacterial agent, desensitizing agents, stain reducing agents, and mixtures thereof.

The expression “mucoadhesive polymer” as used in the specification and claims includes within its meaning hydrophilic polymers and hydrogels. Some polymers useful in the practice of the current invention include: cellulose derivatives, polyvinylpyrrolidones, polyacrylates, polyethers, polyanhydrides, polysaccharides, polyvinylphosphates, and copolymers incorporating these functionalities.

A preferable class of mucoadhesive polymers are the polycarboxylates. The term polycarboxylate means oligomers or polymers with repeat units that have carboxylate functional groups. A particularly preferred mucoadhesive polymer is a copolymer of methylvinyl ether and maleic anhydride known as GANTREZ®, a well known commercially available copolymer, or GANTREZ® AN, a copolymer of polymethyl vinyl ether and maleic anhydride (PVM/MA). The Gantrez polymers are commercially available from ISP Technologies, Inc., Bound Brook, N.J. 08805. Gantrez polymers have been known and used in oral care preparations, and are described, inter alia, in U.S. Pat. Nos. 4,521,551, 4,373,036, and 4,485,090, the disclosures of each of which are incorporated by reference herein in their entirety. Gantrez polymers are advantageous due to its long residence time at enamel surfaces and its ability to deter bacterial adhesion to enamel. Used in combination with an insoluble or slightly soluble metal compound, Gantrez may place a source of metal ions in spatial proximity to the tooth while maintaining a longer residence time of the metal compound in proximity to the tooth.

A mucoadhesive polymer such as a polycarboxylate may be incorporated into the orally acceptable vehicle of the present invention in amounts in the range of 0.01 to 20% by weight, preferably 0.1 to 10% by weight and most preferably at 0.5 to 7% by weight of the component. Mixtures of the mucoadhesive polymers may also be used. Gantrez is the preferred mucoadhesive polymer.

The expression “metal compound or salt” as used in the specification and claims includes within its meaning salts and compounds of zinc. These salts and compounds include zinc oxide, zinc carbonate, zinc citrate, zinc silicate, zinc stannate, zinc benzoate, zinc tetrafluoroborate, zinc hexafluorosilicate, and other zinc compounds. The zinc compound preferably is zinc oxide.

Some insoluble zinc compounds have the ability to react with acid to form zinc ions in solution. Zinc oxide has the ability to react with acid to form zinc ions in solution:

ZnO+HCl→Zn²⁺+2Cl⁻+H₂O

This reaction consumes acid to yield a zinc salt and water. The pH of ZnO is approximately 6.95, which indicates that in an environment that is more acidic (lower pH) than 6.95, the ZnO will dissolve, consuming acid and raising the pH.

The expression “insoluble or slightly soluble” as used in the specification and claims refers to the solubility of the metal salts and compounds. It is known that pH may affect the solubility of compounds that may make these compounds more or less soluble at different pH. It also is understood that solubility encompasses a dynamic equilibrium between precipitation and dissolution which may be affected by factors including but not limited to the presence of soluble chelating agents or acids. Insoluble or slightly soluble can be understood to mean compounds which are less than 1% soluble by weight in pH 7 water. It also will be understood that at lower or higher pH, the compounds may become significantly more soluble, and that the phrase “a metal compound or salt that becomes more soluble at acidic pH” will refer to an insoluble or slightly soluble compound that may become more soluble upon lowering of the local pH, preferably zinc oxide.

An alkaline agent such as an alkali metal compound including sodium hydroxide, potassium hydroxide, sodium bicarbonate, sodium carbonate, N-sodium silicate (a 3.22 weight ratio of sodium silicate in 34.6% water available from PQ Corporation) may be incorporated in the orally acceptable vehicle of the present invention in amounts in the range of 0.5 to 15% by weight, preferably 1 to 8% by weight and most preferably at 1 to 5% by weight of the component. Mixtures of the above alkali metal compounds may also be used. Sodium hydroxide is the preferred alkaline agent.

Abrasives may be incorporated in the orally acceptable vehicle of the present invention and preferred abrasives are siliceous materials, such as silica. A preferred silica is a precipitated amorphous hydrated silica, such as Sorbosil AC-35, marketed by Crosfield Chemicals, or Zeodent 115 from Huber Company but other abrasives may also be employed, including hydroxyapatite, sodiummetaphosphate, potassium metaphosphate, tricalcium phosphate, calcium phosphate dihydrate, anhydrous dicalciumphosphate, calcium pyrophosphate, magnesium orthophosphate, trimagnesium phosphate, calcium carbonate, sodium bicarbonate, alumina trihydrate, aluminum silicate, calcined alumina, titania, and bentonite. The concentration of abrasive in the toothpaste compositions of the present invention will normally be in the range of 5 to 40% by weight and preferably 10 to 25% by weight.

A humectant used in the preparation of the orally acceptable vehicle may be a mixture of humectants, such as glycerol, sorbitol and a polyethylene glycol of molecular weight in the range of 200 to 1000, but other mixtures of humectants and single humectants may also be employed. The humectant content is in the range of 10 to 50% by weight and preferably 20 to 40% by weight of the dentifrice component. The water content is in the range of 20 to 50% by weight and preferably 30 to 40% by weight.

Thickeners used in the preparation of the orally acceptable vehicle include organic and inorganic thickeners. Inorganic thickeners which may be included in the orally acceptable vehicle include amorphous silicas. An inorganic thickener may be incorporated in the orally acceptable vehicle of the present invention at a concentration of 0.5 to 5% by weight and preferably 1 to 3% by weight.

Organic thickeners of natural and synthetic gums and colloids may also be used to prepare the orally acceptable vehicle of the present invention. Examples of such thickeners are carrageenan (Irish moss), xanthan gum, sodium carboxymethyl cellulose, starch, polyvinylpyrrolidone, hydroxyethylpropylcellulose, hydroxybutyl methyl cellulose, hydroxypropyl methyl cellulose, and hydroxyethyl cellulose. An organic thickener may be incorporated in the orally acceptable vehicle of the present invention at a concentration of 0.1 to 3% by weight and preferably 0.4 to 1.5% by weight.

Surfactants may be incorporated in the orally acceptable vehicle to provide foaming properties. The surfactant is preferably anionic or nonionic in nature. Suitable examples of anionic surfactants are higher alkyl sulfates such as potassium or sodium lauryl sulfate which is preferred, higher fatty acid monoglyceride monosulfates, such as the salt of the monosulfated monoglyceride of hydrogenated coconut oil fatty acids, alkyl aryl sulfonates such as sodium dodecyl benzene sulfonate, higher fatty sulfoacetates, higher fatty acid esters of 1,2 dihydroxy propane sulfonate. The surfactant agent may generally be present in the orally acceptable vehicle compositions of the present invention at a concentration of 0.5 to 10% by weight and preferably 1.0 to 5.0% by weight.

The source of desensitizing potassium ion may generally be a water soluble potassium salt including potassium nitrate, potassium citrate, potassium chloride, potassium bicarbonate and potassium oxalate with potassium nitrate being preferred. The potassium salt is generally incorporated in one or more of the dentifrice components at a concentration of 1 to about 20% by weight and preferably 3 to 10% by weight.

Pyrophosphate salts having anticalculus efficacy useful in the practice of the present invention include water soluble salts such as dialkali or tetraalkali metal pyrophosphate salts such as Na₄P₂0₇ (TSPP), K₄P₂0₇, Na₂P₂0₇, Na₂H₂P₂0₇ and K₂H₂P₂0₇. Polyphosphate salts include the water soluble alkali metal tripolyphosphates such as sodium tripolyphosphate and potassium tripolyphosphate. The pyrophosphate salts may be incorporated in the dentifrice composition of the present invention at a concentration of 0.5 to 2% by weight, and preferably 1.5 to 2% by weight and the polyphosphate salts may be incorporated in the dentifrice composition of the present invention at a concentration of 1 to 7% by weight.

Colorants such as pigments and dyes may be used in the practice of the present invention. Pigments include nontoxic, water insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green #1 lake, FD&C Blue #2 lake, FD&C R&D #30 lake and FD&C #Yellow 15 lake. The pigments have a particle size in the range of 5-1000 microns, preferably 250-500 microns, and are present at a concentration of 0.5 to 3% by weight.

Dyes used in the practice of the present invention are generally food color additives presently certified under the Food Drug & Cosmetic Act for use in the food and ingested drugs, including dyes such as FD&C Red No.3 (sodium salt of tetraiodofluorescein), FD&C Yellow No.5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No.3 (disodium slat of 4-{[4-(N-ethyl-p-sulffobenzyno)-phenyl]-(4-hydroxy-2-sulfoniumphenyl)mewthylene}-[1-(N-ethyl-N-p-sulfobenzyl)-G) -3,5cyclohexadienimine], FD&C Blue No.1 (disodium salt of dibenzyldiethyldiaminotriphenylcarbinol trisulfonic acid of indigo tin) and mixtures thereof in various proportions. The concentration of the dye for the most effective result in the present invention is present in the dentifrice composition in an amount from 0.0005 percent to 2 percent of the total weight.

Any suitable flavoring or sweetening material may also be incorporated in the dentifrice composition of the present invention. Examples of suitable flavoring constituents are flavoring oils, e.g., oils of spearmint, peppermint, wintergreen, sassafras, clove, sage, eucalyptus, marjoram, cinnamon lemon, and orange, and methyl salicylate. Suitable sweetening agents include sucrose, lactose, maltose, sorbitol, xylitol, sodium cyclamate, perillatine, and sodium saccharin. Suitably, flavor and sweetening agents may together comprise from 0.01 to 5% or more of the preparations.

Antibacterial agents are non-cationic antibacterial agents based on phenolic and bisphenolic compounds, halogenated diphenyl ethers such as Triclosan, benzoate esters and carbanilides as well as cationic antibacterial agents such as chlorhexidine digluconate. Such antibacterial agents can be present in quantities of from 0.03 to 1% by weight of the particular component.

When noncationic antibacterial agents or antibacterial agents are included in any of the dentifrice components, there is also preferably included from 0.05 to 5% of an agent which enhances the delivery and retention of the agents to, and retention thereof on oral surfaces. Such agents useful in the present invention are disclosed in U.S. Pat. Nos. 5,188,821 and 5,192,531; and include synthetic anionic polymeric polycarboxylates, such as 1:4 to 4:1 copolymers of maleic anhydride or acid with another polymerizable ethylenically unsaturated monomer, preferably methyl vinyl ether/maleic anhydride having a molecular weight (M.W.) of about 30,000 to about 1,000,000, most preferably about 30,000 to about 800,000. These copolymers are available for example as GANTREZ®, e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805. The enhancing agents when present are present in amounts ranging from 0.05 to 3% by weight.

To prepare the dentifrice components of the present invention, generally the humectants, for example, propylene glycol, polyethylene glycol ingredients, are dispersed with any organic thickeners, sweetener, pigments such as titanium dioxide and any polyphosphates included as anti-calculus ingredients. Water is then added into this dispersion along with any antibacterial agent such as Triclosan, any antibacterial enhancing agent such as Gantrez and any anticalculus additional agents. The mucoadhesive polymer and zinc compound or salt that becomes more soluble at acidic pH is then mixed into the dispersion. These ingredients are mixed until a homogenous phase is obtained. Thereafter inorganic thickener, silica abrasive, flavor and surfactant ingredients are added and the ingredients mixed at high speed under vacuum of from about 20 to 100 mm of Hg. The resultant product is a homogeneous, semi-solid, extrudable paste product.

The preparation of dentifrice compositions is well known in the art. U.S. Pat. Nos. 3,996,863, 3,980,767, 4,328,205, and 4,358,437, which are incorporated herein by reference, describe toothpastes and methods of production thereof which may be utilized for the production of the dentifrices according to the present invention.

The term “shear alignment” as used in the specification and claims refers to a process where a material becomes, at least partially, more ordered in response to an applied shear stress. Shear alignment will be understood to create a more homogeneous composition as a result of the at least partial ordering. In the present invention, improved ordering may occur due to the formation of a more regular array of polymer, a more regular array of metal in the polymer, or a combination of both more ordered polymer and metal.

### SPECIFIC EMBODIMENTS OF THE INVENTION

The invention is further described in the following examples. The examples are merely illustrative and do not in any way limit the scope of the invention as described and claimed.

Example—An anti-erosion toothpaste was prepared in which the insoluble zinc compound was ZnO, the source of fluoride was NaF, and high cleaning silica was used as an abrasive. Table 1 contains the ingredients of this anti-erosion toothpaste composition.

TABLE 1
ZnO Formulation

| Ingredient | Weight % |
|---|---|
| DEMINERALIZED WATER | q.s |
| SORBITOL. | 54.9 |
| HIGH CLEANING SILICA Zeo105 | 10.0 |
| SYN. AMORPH. PPT. SILICA | 12.8 |
| POLYETHYLENE GLYCOL | 3.0 |
| SODIUM LAURYL SULFATE POWDER | 1.5 |
| COCAMIDOPROPYL BETAINE | 1.3 |
| FLAVOR | 1.2 |
| ZINC OXIDE | 1.0 |
| TITANIUM DIOXIDE | 0.8 |
| SODIUM CMC\ | 0.7 |
| TETRASODIUM PYROPHOSPHATE | 0.5 |
| SODIUM SACCHARIN | 0.3 |
| SODIUM FLUORIDE | 0.2 |

A modification on the widely used FDA method #34 for the quantification of enamel solubility reduction (ESR) was employed to determine the anti-erosive potential of the formulation described in table 1. This modified method consisted of a one time acid challenge (pH=2.8, citric acid) sustained for 15 minutes after which the amount of dissolved enamel was quantified spectrophotometrically. Upon treating enamel surfaces with the ZnO containing formulation (table 1), and performing the above described evaluation, a 5.6% reduction in enamel solubility was observed. This result indicates the potential benefits of ZnO containing dentifrice formulations with regard to chemically induced demineralization (i.e. erosion) of oral hard tissues.

1. An oral care composition comprising (a) an orally acceptable vehicle (b) a mucoadhesive polymer, (c) a zinc compound or salt that becomes more soluble at acidic pH, and (d) an abrasive wherein the composition provides protection against erosion of tooth enamel.

2. The oral care composition of claim 1 wherein the zinc compound or salt is zinc oxide.

3. The oral care composition of claim 1 wherein the mucoadhesive polymer is a copolymer of a methylvinyl ether and a maleic anhydride.

4. A method of reducing acid based erosion of teeth comprising: 1) providing a composition comprising (a) an orally acceptable vehicle (b) a mucoadhesive polymer, (c) a zinc compound or salt that becomes more soluble at acidic pH, and (d) an abrasive; and 2) delivering the composition to the oral cavity of a subject, wherein delivering the composition deposits a layer of mucoadhesive polymer and zinc compound or salt on tooth enamel.

5. The method of claim 4 wherein the zinc compound or salt is zinc oxide.

6. The method of claim 4 wherein the mucoadhesive polymer is a copolymer of a methylvinyl ether and a maleic anhydride.

7. The method of claim 4, further comprising applying a shear stress to the composition which shear-aligns the layer of mucoadhesive polymer and zinc compound or salt that is deposited on the teeth wherein the shear aligned layer becomes more homogeneous.

8. The method according to claim 7 wherein applying a shear stress results in the mucoadhesive polymer becoming more ordered.

9. The method according to claim 7 wherein applying a shear stress results in the zinc compound or salt becoming more ordered.

10. The method according to claim 4 wherein the zinc compound or salt comprises anisotropically shaped particles.

11. The method according to claim 4 wherein the zinc compound or salt comprises isotropically shaped particles.

12. The method according to claim 7 wherein the shear stress is applied by rubbing the teeth with an apparatus.

13. The method according to claim 12 where the apparatus comprises a toothbrush.
